SEQUENCE LISTING (7 sequences)

SEQ ID NO: 1 - protein, 553 aa, Newcastle disease virus
MGSRPSTKIPAPMMLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 2 - protein, 553 aa, Artificial Sequence
  Synthetic Sequence
MGSRPSTKIPAPMMLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILLLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLFFSRIVTFPMSPGIYSCLSGNTSACMYSKTEGALTTPYMTIKGSVIANCKMTTCRCADPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 3 - protein, 553 aa, Artificial Sequence
  Synthetic Sequence
MGSRPSTKIPAPMMLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETSSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISILDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 4 - protein, 577 aa, Newcastle disease virus
MDRAVSQVALENDEREAKNTWRLIFRIAILLLTVVTLATSVASLVYSMGASTPSDLVGIPTRISRAEEKITSALGSNQDVVDRIYKQVALESPLALLNTETTIMNAITSLSYQINGAANNSGWGAPIHDPDFIGGIGKELIVDDASDVTSFYPSEFQEHLNFIPAPTTGKGCTRIPSFDMSATHDCYSHNVILSGCRDHSHSHQYLALGVLRTTATGRIFFSTLRSISLDDTQNRKSCSVSATPLGCDMLCSKVTGTEEEDYNSAVPTLMVHGRLGFDGQYHEKDLDVTTLFGDWVANYPGVGGGSFIDGRVWFPVYGGLKPNSPSDTVQEGKYVIYKRYNNTCPDEQDYQIRMAKSSYKPGRFGGKRIQQAILSIKVSTSLGEDPVLTVPPNTVTLMGAEGRILTVGTSHFLYQRGSSYFSPALLYPMTVSNKTATRHSPYKFNAFTRPGSPPCQASARCPNSCVTGVYTDPYPLIFYRNHTLRGVFGTMLDSEQARLNPTSAVFDSTSRSRITRVSSSSTKAAYTTSTCFKVVKTNKTYCLSIAEISNTLFGEFRIVPLLVEILKNDGVREARSG

SEQ ID NO: 5 - protein, 6 aa, Artificial Sequence
  Synthetic Sequence
GRQGRL

SEQ ID NO: 6 - protein, 6 aa, Artificial Sequence
  Synthetic Sequence
RRQKRF

SEQ ID NO: 7 - protein, 6 aa, Artificial Sequence
  Synthetic Sequence
RRQRRF

(19) United States
(12) Patent Application Publication
(10) Pub. No.: US 2013/0202558 A1
(43) Pub. Date: Aug. 8, 2013

(54) RECOMBINANT REPLICATION COMPETENT ONCOLYTIC VIRUSES AND METHODS OF USE THEREOF FOR THE TREATMENT OF CANCER

(71) Applicant: James C. Phillips, Piscataway, NJ (US)

(72) Inventor: James C. Phillips, Piscataway, NJ (US)

(21) Appl. No.: 13/757,135

(22) Filed: Feb. 1, 2013

Related U.S. Application Data
(60) Provisional application No. 61/593,831, filed on Feb. 1, 2012.

(51) Int. Cl. A61K 35/76

(52) U.S. Cl. CPC A61K 35/768
USPC 424/93.2

(57) ABSTRACT

Improved NCD variants exhibiting enhanced bioavailability and stability for the treatment of cancer are provided.

This application claims priority to U.S. Provisional Application No. 61/593,831 filed Feb. 1, 2012, the entire contents being incorporated herein by reference as though set forth in full.

### FIELD OF THE INVENTION

This invention relates the fields of molecular biology and virology. More specifically, the invention provides compositions and methods for the rational design of stable, improved oncolytic viruses. Recombinant viruses so produced and methods of use thereof for the treatment of malignant disease are also disclosed.

### BACKGROUND OF THE INVENTION

Several publications and patent documents are cited throughout the specification in order to describe the state of the art to which this invention pertains. Each of these citations is incorporated herein by reference as though set forth in full.

A vaccine is a biological preparation which induces immunity to a particular virus. A vaccine typically contains an agent that resembles a virus, and is often made from weakened or killed forms of the microbe or its toxins. The agent may stimulate the body's immune system to recognize the agent as foreign, destroy it, and “remember” it, so that the immune system can more easily recognize and destroy any of these viruses that it later encounters. In exceptional cases, the agent may selectively attack foreign cells and destroy them. New Castle Disease virus exhibits the ability to lyse infected target cells.

Several prior art references describe New Castle Disease Virus (NDV). NDV strains have been classified as pathogenic (mesogenic or velogenic) or non-pathogenic (lentogenic) to poultry. The 73T, MTH68 and PV701 (MK107) mesogenic strains of NDV have been the subject of several clinical studies. NDV-PV701 has recently been evaluated in a Phase I study of patients with advanced solid tumors; however, patients with CNS tumors were excluded from these studies (Pecora, A. L., et al., J Clin Oncol 20:2251-66, 2002). The anti-neoplastic responses to MTH68 in malignant glioma have been reported (Csatary, L. K., et al. J. Neurooncol. 67: 83-93, 2004).

Lentogenic strains of NDV have also been shown to kill some cancer cell lines (Schirrmacher, V., et al. ibid). Infection of tumor cells by lentogenic NDV has been found to generate several innate danger signals leading to apoptosis. The lentogenic Ulster strain of NDV has been combined with various tumor cells as a tumor vaccine for different cancers including glioblastoma, however the use of a lentogenic NDV strain alone in virotherapy has not been evaluated.

WO 00/62735 of Pro-Virus discloses the use of any interferon sensitive strain of virus for killing neoplastic cells that are deficient in the interferon response. The Pro-Virus disclosure supplies a catalog of viral strains including three mesogenic strains of NDV (MK107, NJ Roakin, and Connecticut-70726) shown to be useful for treatment of human tumor xenografts in athymic mice. NDV administration to these mice caused tumor regression, which was attributed to more efficient and selective replication of NDV in tumor cells versus normal cells. The differential sensitivity of tumor cells to killing by NDV was disclosed to be correlated to an inability of the cells to manifest interferon-mediated antiviral response. The above patent application claims methods of infecting neoplasms or tumors and methods of treating neoplasms or tumors by interferon-sensitive, replication competent RNA or DNA viruses.

European Patent No. 0696326 discloses a use of NDV in manufacturing of a medicament for treatment of cancer, wherein the NDV is of moderate virulence and is cytolytic. European Patent Application No. 1314431 discloses a composition comprising NDV for use in the treatment of cancer, wherein the NDV is of moderate virulence and is cytolytic. European Patent Application No. 01486211 claims a use of NDV in the manufacture of medicament for treatment of cancer in a mammal having a tumor wherein the medicament is administered systematically in multiple doses to said mammal in an amount sufficient to cause tumor regression. Though European Patent Application No. 01486211 refers to various strains of NDV, both cytolytic and non-cytolytic, the applicants of the European application provide in vivo effects of two mesogenic strains of NDV in animal models. No clinical studies nor examples of in vivo effects of lentogenic strains of NDV are disclosed in the European Patent Application No. 01486211. International Patent Application WO 2005/018580 claims a method of treating a mammalian subject having a tumor comprising administering to the subject an amount of a NDV. Though the NDV according to WO 2005/018580 can be of low (lentogenic), moderate (mesogenic) or high (velogenic) virulence, the application relates to a mesogenic strain of NDV. No clinical studies nor examples of in vivo effects of lentogenic strains of NDV are disclosed in WO 2005/018580.

International Patent Application WO 2003/022202 discloses pharmaceutical compositions comprising a lentogenic oncolytic strain of NDV and methods for treating cancer comprising same. International Patent Application WO 2003/022202 discloses pre-clinical studies that demonstrate the oncolytic activity of a lentogenic strain of NDV.

U.S. Patent Application Publication No. 2004/0131595 discloses a method for treating a mammalian subject having a carcinoid tumor comprising administering to the subject a negative-stranded RNA virus. The negative-stranded RNA virus is a replication competent oncolytic virus, particularly a NDV, and more particularly a mesogenic strain of NDV.

While NDV has been disclosed for use in the treatment of cancer, the strains currently under study still suffer from certain drawbacks. None of these strains have been tested in clinical trials beyond Phase I. Clearly, a need exists in the art for improved oncolytic viruses that can be used for the treatment of currently incurable cancers.

### SUMMARY OF THE INVENTION

It is an object of the present invention to provide methods for the rational design of potent recombinant oncolytic viruses, recombinant viruses so produced and methods of use thereof for the treatment of malignant disease. In one embodiment variant Newcastle Disease Viruses are provided which have been designed to include certain genetic alterations that improve in vivo availability and stability. The variants may also exhibit enhanced oncolytic action when compared to virus strains encoding proteins lacking the alterations described herein. The improved NDV variants can be administered to a patient in need thereof via arterial infusion and do not require direct intratumor injection as previously reported in other studies employing NDV in anti-cancer treatments. NDV proteins are similar to influenza proteins, except that their glycosidic proteins are qualitatively different. Thus the hemagglutinin (HA) and neuraminidase (NA) influenza glycosidic proteins are replaced in NDV by the hemagglutinin—neuraminidase (HN) and fusion (F) glycosidic proteins. We disclose rational designs of the NDV HN and F glycosidic proteins that may also exhibit enhanced oncolytic action when compared to virus strains encoding proteins lacking the alterations described herein. It is also expected that the present rationally designed virus strains will exhibit reduced virulence against healthy tissues, enabling delivery of massive doses by arterial injection. The methods used to design these glycosidic proteins may also be extended to other NDV proteins, such as the nonstructural protein NS1 which regulates interferon activity.

### BRIEF DESCRIPTION OF THE DRAWINGS

FIG. 1 provides a flow chart of the process used to design the improved oncolytic viral variants of the invention.

FIG. 2 is an example of hydroprofiles of several reference wild NDV F proteins with W=111, plotted against amino acid site number, using the MZ hydropathicity scale. The differences due to evolution are small, and are best seen near the N terminal (small site numbers <100). The hydroprofile of the synthetic NDV F Design 1 is also shown. As described in FIG. 1, the smoothness of Design 1 has been increased relative to the wild NDV F proteins by choosing from the NCBI data base RC mutations that lower hydroprofile peaks and raise valleys. Peak lowering is clearly visible between sites 280 and 320, while valley raising is seen near 100 and near 440. The largest differences are seen near the cleavage site 117 and between the third (366) and fourth (447) glycosylation sites. The heptad repeat framework regions HRA and HRB (Swanson et al. (2010) Virol. 402:372-379) are largely conserved between historic strains. However, in NDV F there is also an HRA near 280 (McGinnes et al. (2001) Virol. 289:343-352).

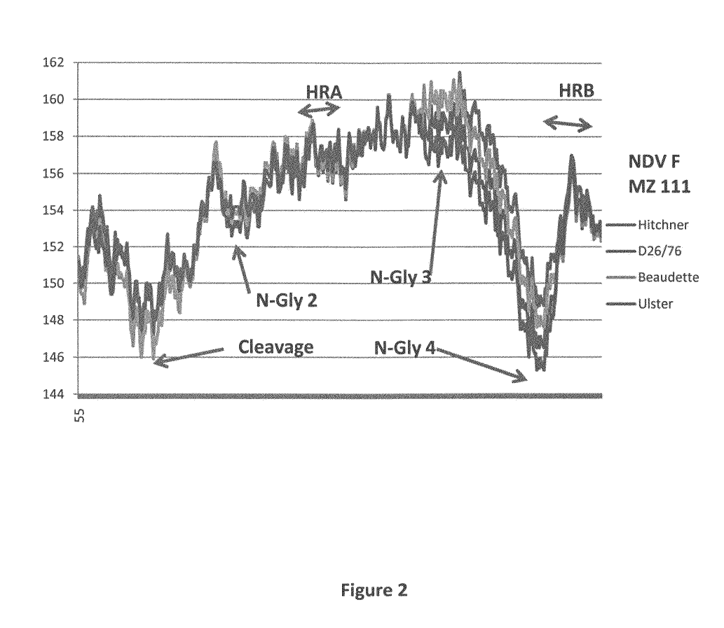

FIG. 3 illustrates the smoothing of the NDV F Hitchner W=111 modular profile by design mutations D1, D3 and D5 (see Table I for definitions of these designs). It is not obvious from this figure how much the designs improve the smoothness of the profile, but actual calculations show large improvements (Table I). Roughness has been proposed as a key factor determining the strength of protein-protein dimerizing (or oligomerizing) interactions, but previous work has not permitted explicit calculations of the effects of mutations on roughness for large proteins containing hundreds of amino acids (Yu, N.; Hagan, F. (2012) Simulations of HIV Capsid Protein Dimerization Reveal the Effect of Chemistry and Topography on the Mechanism of Hydrophobic Protein Association. Biophys. J. 103: 1363-1369). Note that the smoothing of D5 relative to Hitchner occurs by smoothing the central region 280 to 370, as well as balancing it against the region 140-200, which buttresses the conserved fusion peptide 117-140 (a secondary hydrophobic peak, related to a conserved, structurally significant heptad L repeat sequence). Surprisingly, most of the changes engineered by splicing different standard strains to increase smoothness occur in the regions (such as 220-380) that are nearly conserved in the historic strains shown in FIG. 2.

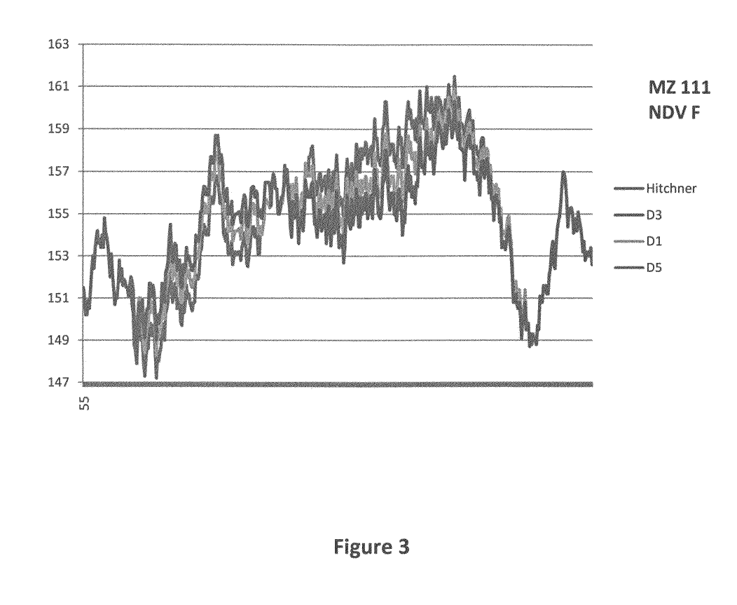

FIG. 4 presents hydroprofiles of historic NDV HN strains. These are qualitatively different from those of NDV F or HA1 and NA1 of common flu H1N1. Such profiles are used according to the flow diagram of FIG. 1 to obtain the mutations listed in Tables III (MZ scale) and IV (KD scale). A pronounced hydrophilic central hinge is present near 300, between the 119 and 341 N-glycan sites. With high resolution one can see that the largest strain differences, which smooth Beaudette and Hitchner compared to Italien and Miyadera, occur below 150 nearer the N terminal.

FIG. 5 is a sketch, based on a figure from (Ebert 2010), of the anticipated in vivo effects of using our F Design 1 or 2 strain NDV viruses on rats implanted with liver cancers, compared to his strains based on L289A mutation. The comparative advantages of our mutations are evident. Even larger effects, approaching complete remission, are anticipated when both F Design 1 or 2 and HN Design α or β strain NDV are applied. Such large effects of our designed NDV may also be observed in vivo for humans, because our RC designs are based only on isolated mutations of wild NDV contained in the NCBI NDV data base.

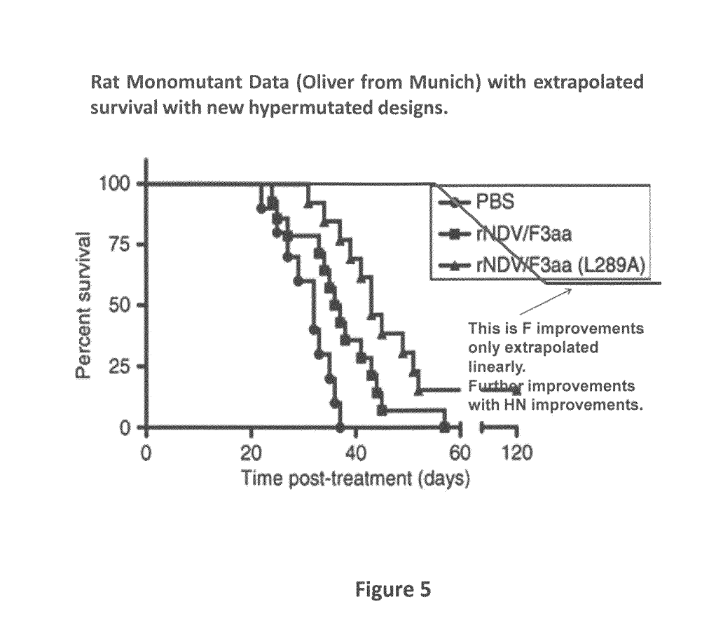

### DETAILED DESCRIPTION OF THE INVENTION

### Accelerated Scaling Method for Design of Optimal Viral Cancer Cell Killing Proteins

Modern methods of characterizing viruses and their encoded viral proteins have given rise to large-scale sequence data bases, (e.g., the NCBI Influenza and NDV Virus Sequence and Nucleotide Database). In accordance with the present invention, methods are provided for generating improved viral compositions via an appropriately applied general sequence scaling method. Variant viruses so produced and methods of use thereof for the treatment of disease are also provided.

In accordance with the present invention, improved, recombinant NDV variants for use in oncolytic viruses for the treatment of cancer are disclosed. Also provided are methods employing such viruses to induce tumor regression in cancer patients. In a preferred embodiment, the patients are stage 4 cancer patients for whom previous therapies have been unsuccessful. The compositions of the invention are non-pathological and non-poisonous, and have been shown to produce only minor side effects, and so may be preferred to other therapies at all cancer stages.

The effectiveness of the therapy can be monitored by assessing expression levels of certain biomarkers associated with malignancy. An exemplary method entails obtaining a biologicial specimen from the patient before, during and after cancer treatment to determine whether the treatment has altered informative biomarker expression thereby indicating the efficacy of the treatment applied and providing the clinician with guidance as to whether further treatment is required. Such markers can include, without limitation, in pancreatic cancers, a microRNA such as miR-18a (Morimura, R., et al., British J. Cancer 105, 1733 (2011)), or in colon cancers miR-92a (Tsuchida, A., et al., Cancer Sci. 102, 2264-2271 (2011), or in prostate cancers miR-141 (Gonzales, J. C., et al., Clinical Gen. Cancer 9, 39 (2011), or in liver cancers miR-122 and miR-192 (Wang, K., et al., Proc. Nat. Acad. Sci. (USA) 106:4402 (2009)), or similar biomarkers.

### I. Definitions

The following definitions are provided to facilitate an understanding of the present invention. Unless defined otherwise, all technical and scientific terms used herein have the same meaning as commonly understood by one of ordinary skill in the art to which this invention belongs. Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); RNA Viruses: A Practical Approach, (Alan, J. Cann, Ed., Oxford University Press, 2000); and Huiping et al., Analytical Biochemistry Volume: 418 Issue: 2 Pages: 304-306. A commercial kit facilitating Site-Directed Mutagenesis is available: Stratagene QuikChange™.

For purposes of the invention, “Nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule, called a strain, may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production. Here we describe mutated DNA or RNA sequences in terms of mutated amino acid sites, which can be converted to partner nucleotide sequences by standard methods, or by referring to the partner sequences in the NCBI NDV data base.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein the term “plaque-forming unit” (pfu) means one infectious virus particle.

As used herein the term “multiplicity of infection” (MOI) means the number of infectious virus particles added per cell.

As used herein the term “clonal virus” means a virus derived from a single infectious virus particle and for which individual molecular clones have significant nucleic acid sequence homology. For example, the sequence homology is such that at least eight individual molecular clones from the population of virions have a sequence homology greater than 95% over 300 contiguous nucleotides.

As used herein “NDV” is an abbreviation for Newcastle Disease Virus.

As used herein, the term “regression” of a tumor means decreasing tumor size or arresting tumor growth or tumor progression, which have their commonly understood meaning of suppressing tumor growth.

The term “oncolytic virus” as used herein refers to a virus capable of exerting a cytotoxic or killing effect in vitro and in vivo to tumor cells with little or no effect on normal cells. The term “oncolytic activity” refers to cytotoxic or killing activity of a virus to tumor cells. Without wishing to be bound to any mechanism of action, the oncolytic activity exerted by a lentogenic strain of NDV, particularly the recombinant strains described herein, is probably primarily due to cell apoptosis and to a lesser extent to plasma membrane lysis, the latter is accompanied by release of viable progeny into the cell's milieu that subsequently infect adjacent cells. The cytotoxic effects under in vitro or in vivo conditions can be detected by various means as known in the art, for example, by inhibiting cell proliferation, by detecting tumor size using gadolinium enhanced MRI scanning, by radiolabeling of a tumor, and the like.

For clinical studies, it is desirable to obtain a clonal virus so as to ensure virus homogeneity. Clonal virus can be produced according to any method available to the skilled artisan. For example, clonal virus can be produced by limiting dilution or by plaque purification. A series of cloned lentogenic NDV strains denoted by SEQ ID NOS: are fully set forth herein. Methods for purifying the NDV strains of the invention are disclosed in International Patent Application WO 2003/022202, for example, wherein the clonal NDV HUJ strain was prepared by limiting dilution and further purified on a sucrose gradient.

All types of tumors accessible via arterial infusion can be treated using the oncolytic viral formulations of the invention. As a non limiting example, the following solid tumors can be treated: skin (e.g., squamous cell carcinoma, basal cell carcinoma, or melanoma), colorectal, prostate, head and neck, testicular, ovarian, pancreatic, liver (e.g., hepatoma), kidney, bladder, gastrointestinal, endocrine system (e.g., thyroid and pituitary tumors), and lymphatic system (e.g., Hodgkin's and non-Hodgkin's lymphomas) tumors.

The methods of the invention can be used to induce regression of primary tumors and tumor metastases. The NDV administered according to the methods of the invention follow the same pathways as metastasizing tumor cells, thus enhancing the likelihood of NDV reaching those areas within the lymphatic system, e.g., lymph nodes that are at greatest risk for harboring metastatic disease.

The pharmaceutical compositions of the invention comprise as an active ingredient a lentogenic oncolytic strain of NDV, particularly the variant NDV strains disclosed herein in a form suitable for administration to a human subject. The pharmaceutical compositions can further comprise one or more pharmaceutically acceptable carriers, one or more additional ingredients, or a combination thereof.

As used herein the term “replication-competent” virus refers to a virus that produces infectious progeny in cancer cells.

The term “vector” relates to a single or double stranded circular nucleic acid molecule that can be infected, transfected or transformed into cells and replicate independently or within the host cell genome. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. An “expression vector” is a specialized vector that contains a gene or nucleic acid sequence with the necessary regulatory regions needed for expression in a host cell. Here we also use the phrase “Replication Competent” or RC to describe viruses that are vectors.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, “pharmaceutical formulations” include formulations for human and veterinary use which exhibit no significant adverse toxicological effect. The phrase “pharmaceutically acceptable formulation” as used herein refers to a composition or formulation that allows for the effective distribution of the compositions of the instant invention in the physical location most suitable for their desired activity. The phrase “pharmaceutically acceptable” is used to indicate that the carrier can be administered to the subject without exerting significant adverse toxicological effects. The term “therapeutically effective amount” is the amount present that is delivered to a subject to provide the desired physiological response (e.g., viral load reduction). Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

The term “treating” or “to treat” as used herein means activity resulting in the prevention, reduction, partial or complete alleviation or cure of a disease or disorder. The term “modulate” means altering (i.e., increasing or decreasing) the biological activity of a system.

“Corresponding” means identical to or complementary to the designated sequence. The sequence may be generated in any manner, including chemical synthesis, DNA replication, reverse transcription or a combination thereof. Being “Complementary” means that a nucleic acid, such as DNA and RNA, encodes the only corresponding base pair that non-covalently connects sequences by two or three hydrogen bonds. There is only one complementary base for any of the bases found in DNA and in RNA, and skilled artisans can reconstruct a complementary strand for any single stranded nucleic acid.

The phrase “consisting essentially of when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “subject” or “patient” includes, but is not limited to animals, including mammalian species such as murine, porcine, ovine, bovine, canine, feline, equine, human, and other primates.

The phrase “viral load” is a measure of the level of a viral infection, and can be calculated by estimating the amount of virus in a cell or patient.

A “derivative” of a polypeptide, polynucleotide or fragments thereof means a sequence modified by varying the sequence of the construct, e.g. by manipulation of the nucleic acid encoding the protein or by altering the protein itself. Such derivatives of the natural sequence may involve insertion, addition, deletion or substitution of one or more amino acids, and may or may not alter the essential activity of original the polypeptide. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide can depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in site-directed mutagenic applications using Quikchange, the oligonucleotide primer is typically 25-30 or more nucleotides in length (Wang H., et al., Analytical Biochem. 418, 304-306 (2011)). The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195; 4,800,195; and 4,965,188, the entire disclosures of which are incorporated by reference herein. The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, depending on the complexity of the target sequence, the oligonucleotide probe typically contains about 10-50 or more nucleotides, more preferably, about 15-25 nucleotides.

The terms “percent similarity”, “percent identity” and “percent homology” when referring to a particular sequence are used as set forth in the University of Wisconsin GCG software program. The term “delivery” as used herein refers to the introduction of foreign molecule (i.e., protein containing nanoparticle) into cells.

The term “administration” as used herein means the introduction of a foreign molecule into a cell. The term is intended to be synonymous with the term “delivery”. Administration also refers to screening assays of the invention (e.g., routes of administration such as, without limitation, intravenous, intra-arterial, intramuscular, subcutaneous, intrasynovial, infusion, sublingual, transdermal, oral, or topical).

The term “kit” refers to a combination of reagents and other materials.

### II. Therapeutic Uses of NDV Variants of the Invention

The NDV variants may be used according to this invention, for example, as therapeutic agents that induce tumor regression in cancer patients by triggering a lytic reaction in the target cell. In a preferred embodiment of the present invention, the NDV variants may be administered to a patient via arterial infusion in a biologically compatible carrier. The NDV variants may be administered alone or in combination with other agents known to have anti-cancer effects. An appropriate composition in which to deliver NDV variants may be determined by a medical practitioner upon consideration of a variety of physiological variables. Nucleic acid molecules encoding the NDV variants of the invention may be prepared by using recombinant DNA technology methods. The availability of nucleotide sequence information enables preparation of nucleic acid-based molecules of the invention by a variety of means.

### III. Pharmaceutical Compositions

The NDV variants of the present invention may be incorporated into pharmaceutical compositions that may be delivered to a subject. In a particular embodiment of the present invention, pharmaceutical compositions comprising purified virus for delivery to a recipient are provided. The compositions may be administered alone or in combination with at least one other agent, such as a stabilizing compound, which may be administered in any sterile, biocompatible pharmaceutical carrier, including, but not limited to, saline, buffered saline, dextrose, and water. In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol.

Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. (1990).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

After pharmaceutical compositions have been prepared, they may be placed in an appropriate container or kit and labeled for treatment. For administration of NDV oncolytic virus, such labeling would include amount, frequency, and method of administration.

Determining a therapeutically effective dose is well within the capability of a skilled medical practitioner using the techniques provided hereinbelow. Therapeutic doses will depend on, among other factors, the age and general condition of the subject, and the site and the severity of the. Thus, a therapeutically effective amount in humans will fall in a relatively broad range that may be determined by a medical practitioner based on the response of an individual patient to the vaccine.

### IV. Kits and Articles of Manufacture

Any of the aforementioned compositions or methods can be incorporated into a kit which may contain at least one NDV variant virus. If the pharmaceutical composition in liquid form is under risk of being subjected to conditions which will compromise the stability of the virus it may be preferred to produce the finished product containing the virus in a solid form, e.g. as a freeze dried material, and store the product is such solid form. The product may then be reconstituted (e.g. dissolved or suspended) in a saline or in a buffered saline ready for use prior to administration.

Hence, the present invention provides a kit comprising (a) a first component containing at least one NDV variant virus as defined hereinabove, optionally in solid form, and (b) a second component containing saline or a buffer solution (e.g. buffered saline) adapted for reconstitution (e.g. dissolution or suspension) or delivery of said virus.

Preferably said saline or buffered saline has a pH in the range of 4.0-8.5, and a molarity of 20-2000 mM. In a preferred embodiment the saline or buffered saline has a pH of 6.0-8.0 and a molarity of 100-500 mM. In a most preferred embodiment the saline or buffered saline has a pH of 7.0-8.0 and a molarity of 120-250 mM. For one embodiment of a kit, the NDV variant comprises a sequence provided herein.

### V. Clinical Applications

As mentioned previously, a preferred embodiment of the invention comprises delivery of at least NDV variant virus to a patient in need thereof. Formulation, dosages and treatment schedules have also been described hereinabove. Phase I clinical trials can be carried out with the informed consent of patients. Phase I clinical trials can be designed to assess the safety, tolerability, pharmacokinetics, and pharmacodynamics of the viral compositions of the invention. These trials may be conducted in an inpatient clinic, where the subject suffering from cancer and can be observed by full-time medical staff. After the initial safety of the therapy has been performed, Phase II trials can assess clinical efficacy of the therapy; as well as to continue Phase I assessments in a larger group of volunteers and patients. Subsequently, Phase III studies on large patient groups entail definitive assessment of the efficacy of the engineered virus for treatment of cancer in comparison with current treatments. Finally, Phase IV trials involving the post-launch safety surveillance and ongoing technical support for the treatment described can be completed.

The following Examples are provided to illustrate certain embodiments of the invention. They are not intended to limit the invention in any way.

### EXAMPLE I

### Evolution of Normal Influenza H1N1 Variants and Designation of Newcastle Disease Viral (NDV) Variants Exhibiting Improved Oncolytic Properties

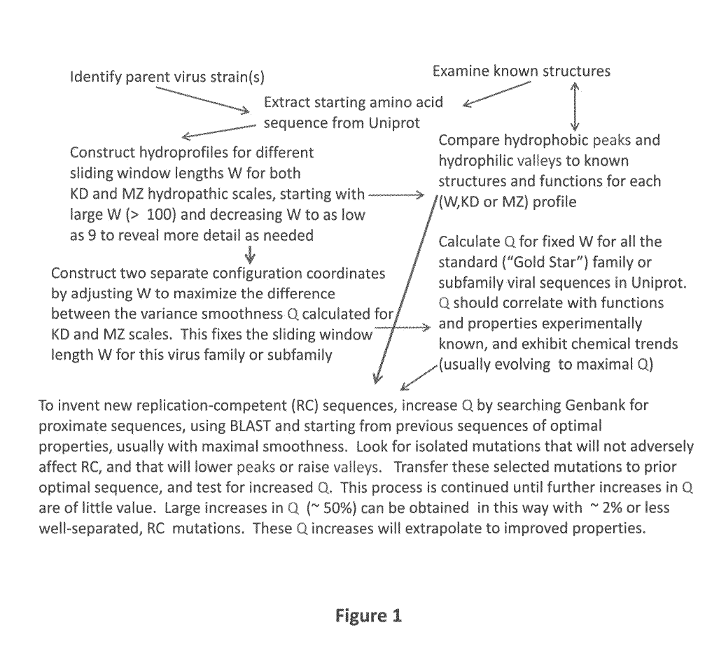

Sequence information available for normal influenza H1N1 HA and NA, and NDV viral glycoproteins HN and F will be used to illustrate the scaling method employed to generate improved viral compositions of the invention. A schematic diagram of the process is provided in FIG. 1. Five influenza viral genome segments have maintained an unbroken evolutionary history within humans-those encoding the nucleocapsid protein (NP), the matrix proteins (M1 and M2) and the nonstructural proteins (NS1 and NS2), and two encoding polymerase proteins (PB2 and PA). In contrast, new haemagglutinin (HA) and neuraminidase (NA) surface glycoproteins, as well as the PB1 polymerase, have been acquired by human influenza A virus through reassortment with avian influenza viruses. NDV proteins are similar to influenza proteins, except that their glycosidic proteins are qualitatively different. Thus the hemagglutinin (HA) and neuraminidase (NA) influenza glycosidic proteins are replaced in NDV by the hemagglutinin—neuraminidase (HN) and fusion (F) glycosidic proteins. The present scaling method identifies associated NDV HN and F protein strains which are more suitable for use as targets than previously employed strains. The examples used here shows how to choose between HA and NA associated influenza viral H1N1 glycoproteins, as well as NDV HN and F glycoproteins. The scaling method is applied to HN and F protein sequences obtained from public data bases such as the NCBI H1N1 and NDV Virus Sequence Database or Uniprot.

Antibodies elicited by different viral proteins have distinct properties in immunity against a given virus. For example, antibodies to influenza HA generally neutralize viral infectivity by interference with virus binding to sialic acid receptors on the target cells or, subsequently, by preventing the fusion of the viral and cellular membranes through which the viral genome gains access to the target cell. Antibodies to NA disable release of progeny virus from infected cells by inhibiting the NA-associated receptor-destroying enzymatic activity. At the same time, HA-NA balance confers respiratory-droplet transmissibility of the pandemic H1N1 influenza virus. The invention utilizes public data bases to identify which viral protein provides the most stable partner among all viral proteins more rapidly and reliably than experimental scans, which may involve high-throughput screening. The remaining viral proteins will adapt to the most stable partner. Similar methods have been used to design optimized sequences for NDV HN and F glycosidic proteins.

The invention utilizes certain computational tools, including multiple amino acid hydropathic scales, that quantify the strength of water-protein interactions. As an example two hydropathic scales are specified which are independent of the target viral proteins. These scales are displayed by J. C. Phillips, Phys. Rev. E 80, 051916 (2009). They are based on different physical mechanisms for describing water-protein interactions. For a given target protein and given property or properties, the invention specifies a scaling method for determining which of these hydropathic scales (or alternative scales) is superior. The determinative scaling method utilizes only target protein sequences from public data bases. No prior method for optimizing viral protein sequences has utilized hydropathic scales.

Hydropathic scales have previously been described in the comparison of non-viral protein sequences, either wild or mutated (J. C. Phillips (2009); arXiv 1102.2433; 1109.2629; 1101.2923. The first scale, denoted by KD, is believed to be better suited to first-order (stronger) interactions with protein antigens or membrane fusion, while the second scale, denoted by MZ, best describes second-order (weaker) interactions occurring during evolution of viral proteins caused by interactions with vaccines and/or survival from injection or transcription site to cancer target.

Viral protein activity depends on many factors. The invention focuses on quantifying one of these, the hydropathic roughness R (or the hydropathic smoothness S=1/R) at the molecular scale of the water—viral protein interface (J. C. Phillips, arXiv 1102.2433; 1109.2629;1101.2923). Among possible viral protein targets, the one that evolves nearly monotonically to become smoothest is selected as the preferred target. Smoothness appears to be the dominant factor in determining viral efficacy (J. C. Phillips, rhodopsin disease mutations, arXiv 1201.1041). There are two possible reasons for this: first, given the tumbling motions that accompany all protein translation after injection or transcription, the smoother globular strains will be more likely to survive to reach their targets. Second, having reached their targets, the globules must still go through a multiplicity of conformations before completing their first task (such as membrane fusion following oligomer formation, in the example of viral proteins). These local evolutions will be completed more rapidly, with a higher probability of success, for smoother proteins. For many membrane proteins, these local evolutions involve oligomer formation, which proceeds more rapidly and more successfully for smoother strains. The smoothness mechanism has been tested against the evolution (1918-present) of H1N1 influenza glycoproteins HA and NA (see below), and against allele controller/progressor statistics of HIV MHC proteins (Int HIV Controllers Study, Science 330, 1551-1557 (2010); results not shown here). In the example of the flu HA and NA glycoproteins, HA is antigen-variable, and does not evolve to become smooth, but NA does; thus NA is the preferred flu protein target. If more than one viral protein evolves nearly monotonically to become smooth, the protein exhibiting the largest fractional decrease in hydropathic roughness is typically selected as the target protein. Alternatively, the protein whose activity is most affected by altering the smoothness is selected.

The calculation of the roughness R=1/S, where S is the smoothness, depends on the choice of a sliding window length scale W. This choice is made from objective criteria independent of the strain sequence being mutated. For example, W can be chosen as the value that maximizes the difference between the values of R calculated from the MZ and KD scales. Another choice focuses on characteristic modular lengths of important functional elements, such as transmembrane (TM) region and the adjacent stalk regions, or silac acid contact points, or glycosidic spacing. Whatever the approach, once W is fixed, a comprehensive survey of many mutated strains and their property differences is determined, thereby providing an accurate and objective accelerated scaling method for designing optimal viral proteins exhibiting oncolytic characteristics.

The invention enables analysis of the evolution of the target viral protein roughness R region-by-region by constructing hydropathic profiles. An example of a hydropathic profile is FIG. 1, J. C. Phillips arXiv 1101.2923, which shows the effect of pressure on lamprey amino acid sequence hydropathic profiles. In the H1N1 NA example, the regions which have evolved most since the 1918 flu epidemic are near the N terminal region. The human 1918 NA1 shares many sequence and structural characteristics with avian strains, including the conserved active site, wild-type stalk length, glycosylation sites, and antigenic sites. Phylogenetically the 1918 NA1 gene appears to be intermediate between mammals and birds, suggesting that it was introduced into mammals just before the 1918 pandemic. The transmembrane (TM) region and the adjacent stalk region are near the N terminal. Evolutionary changes in the roughness R are mainly due to amino acid mutations in the TM and stalk regions. The methodology provided herein reveals that these wild NA1 regions have become smoother since 1918 in three punctuated stages: 1944-1976; 1978-2000; and 2007-present, with two interruptions 1976 (the Fort Dix outbreak), and 2001-2007 (swine flu).

The details of the evolution of NA1 are given in J. C. Phillips arXiv 1209.2616. The general features of influenza virus evolution are described more briefly in J. C. Phillips arXiv 1210.0048, where it is suggested that similar results might be obtainable for NDV, but no detailed mutations are described. These are described here for NDV F and NDV HN for the first time.

The invention possesses several comparative advantages. For H1N1 influenza proteins it generates a synthetic viral protein which can replace or supplant existing viral proteins. In the NA1 example, the synthetic protein is smoother than either its partially avian or partially swine viral antecedents, and can be less infectious than either. It produces a more effective virus, because smoother mutants are more easily and reliably transported by the circulatory system.

The design or engineering step is maximally accelerated compared to experimental searches, as the analysis can be completed in hours after ten to twenty protein sequences are known for each target viral protein. For even larger numbers of proteins from the NCBI, the invention provides an objective scaling method for selecting optimized proteins from even these larger numbers. By comparing measured properties of a few proteins using their hydropathic properties, it enables interpolation or extrapolation of these properties to the entire available data base as well as synthetic constructs.

Using the scaling method described above, a variety of Newcastle Disease Virus (NDV) variants designed for oncolytic applications have been produced. Due to the improved smoothness methodology employed to generate these variants, they should exhibit enhanced fusogenicity when compared to NDV strain lacking these genetic alterations and thus exhibit enhanced cancer cell killing (CCK) effects. NDV contains many parts, mostly structural. The two parts that are key to fusogenic CCK are glycoproteins, which selectively bind to cancer cell membranes (Echchgadda I., et al., Cancer Gene Therapy 16, 923-935 (2009)) as oligosaccharides (Tappert M. M., et al., J. Virology 85, 12146-12159 (2011); Yuan P. et al., Proc. Nat. Acad. Sci. (USA) 108, 14920-14925 (2011)). These two glycoproteins of NDV are the fusion protein F and the hemagglutinin-neuraminidase (HN) protein, which envelopes F and activates it (Colman P M, et al., Nature Rev. Mol. Cell Biol. 4, 309 (2003); Earp L J, et al., Membrane Trafficking in Viral Repl. 285, 25 (2005)). The disclosed designs enhance the smoothness of both F and HN with multiple mutations. The principles guiding the Newcastle Disease Virus NDV designs have been proved against normal flu glycoproteins hemagglutinin (HA) and neuraminidase (NA), as discussed above, as well as many other proteins.

Examples of the designed NDV proteins follow. The designs start from the “Gold Star” Uniprot NDV reference protein strains designated as Texas g.b./48, Beaudette C/45, Italien/45, Miyadera/51, Kansas, D26/76, Hitchner/47, Ireland/Ulster/67, Chi/85, Her/33, Iba/85, LaSota/46, and Queensland/66. The strain Her/33 is specifically excluded, as it is a statistical outlier and its amino acid and nucleotide sequences may have been corrupted. With respect to the smoothness defined by the two scales MZ and KD, S(MZ) and S(KD), the S(HN) and S(F) coordinates of these reference protein strains form a cluster. In the case of the F protein, the reference protein strain with the largest S for the MZ scale is Hitchner/47, while for the KD scale Miyadera/51 S(F) is slightly larger. As an example, we have used Hitchner/47 (Uniprot P33613) as the starting point for our synthetic mutations, but a second and slightly different series of sequence strains would be obtained by starting from Miyadera/51. The Hitchner/47 wild strain is often used in studies of NDV oncolytic activity (for example, Ebert 2010, U.S. patent application Ser. No. 12/520,571). The differences in S between strains obtained from these two starting points MZ and KD scales are small, and the better choice would depend on the kind of cancer treated. Similarly, which of the two hydropathicity scales, MZ or KD, or possibly which combination of the two scales yields better synthetic NDV's, may depend on the kind of cancer treated.

The Hitchner/47 reference sequence is set forth below (SEQ ID NO: 1):

10 20 30 40 50 60
MGSRPSTKIP APMMLTIRVA LVLSCICPAN SIDGRPLAAA GIVVTGDKAV NIYTSSQTGS
70 80 90 100 110 120
IIVKLLPNLP KDKEACAKAP LDAYNRTLTT LLTPLGDSIR RIQESVTTSG GGRQGRLIGA
130 140 150 160 170 180
IIGGVALGVA TAAQITAAAA LIQAKQNAAN ILRLKESIAA TNEAVHEVTD GLSQLAVAVG
190 200 210 220 230 240
KMQQFVNDQF NKTAQELDCI KIAQQVGVEL NLYLTELTTV FGPQITSPAL NKLTIQALYN
250 260 270 280 290 300
LAGGNMDYLL TKLGIGNNQL SSLIGSGLIT GNPILYDSQT QLLGIQVTLP SVGNLNNMRA
310 320 330 340 350 360
TYLETLSVST TRGFASALVP KVVTQVGSVI EELDTSYCIE TDLDLYCTRI VTFPMSPGIY
370 380 390 400 410 420
SCLSGNTSAC MYSKTEGALT TPYMTIKGSV IANCKMTTCR CVNPPGIISQ NYGEAVSLID
430 440 450 460 470 480
KQSCNVLSLG GITLRLSGEF DVTYQKNISI QDSQVIITGN LDISTELGNV NNSISNALNK
490 500 510 520 530 540
LEESNRKLDK VNVKLTSTSA LITYIVLTII SLVFGILSLI LACYLMYKQK AQQKTLLWLG
550
NNTLDQMRAT TKM

F protein oncolytic activity is often enhanced by replacement of Hitchner 112GRQGRL117 (low virulence Hitchner; SEQ ID NO: 5) by 112RRQKRF117 (high virulence Beaudette C; SEQ ID NO: 6) or 112RRQRRF117 (Park 2006; SEQ ID NO: 7) amino acid sequence. This Beaudette C or Park enhanced cleavage option is well known (Collins et al 1993, Ebert 2010) and is always implicitly included in our designed alternatives described for Hitchner 112GRQGRL117 (SEQ ID NO: 5) but also applicable to Hitchner modified by 112RRQKRF117 (SEQ ID NO: 6) or a 112RRQRRF117 (SEQ ID NO: 7) amino acid sequence (Park 2006).

The present state of NDV F oncolytic art (Ebert 2010) is represented by Hitchner modified by 112RRQRRF117 (SEQ ID NO: 7) and mutated by L289A. This strain has been shown to prolong survival significantly compared with unmutated by L289A NDV of immune-competent Buffalo rats bearing multifocal, orthotopic liver tumors (Ebert 2010), and produce more than 10% remission of the implanted tumors. An estimate of the strength of these beneficial effects is given by combining the S(MZ) and S(KD) smoothnesses into the product Q=S(MZ)S(KD). The improvement of Q of Hitchner modified by 112RRQRRF117 (SEQ ID NO: 7) and mutated by L289A compared to unmutated Hitchner modified by 112RRQKRF117 (SEQ ID NO: 6) is about 8%.

Next we mutate a few F sites according to the flow diagram shown in FIG. 1. This leads to two F designs, one emphasizing improvement of S(MZ), the other emphasizing improvement of S(KD). Design 1 is Hitchner F (which may or may not be modified by 112RRQRRF117) mutated by (V402A, N403D), R153L, and (Y346F, C347F, T348S). The improvement of Q=S(MZ)S(KD) of Hitchner F modified by 112RRQRRF117 (SEQ ID NO: 7) and mutated by Design 1 compared to unmutated Hitchner F modified by 112RRQKRF117 (SEQ ID NO: 6) is about 30%, which is an improvement of Q by nearly a factor of 4 compared to the improvement obtained from L289A. Design 2 is Hitchner F modified by 112RRQRRF117 (SEQ ID NO: 7) and mutated by L3065 and Q451L. The improvement of Q of Hitchner F modified by 112RRQRRF117 (SEQ ID NO: 7) and mutated by Design 2 compared to unmutated Hitchner F modified by 112RRQKRF117 (SEQ ID NO: 6) is about 33%, which is again an improvement of Q by a factor of 4 compared to the improvement obtained fromL289A.

There is an additional comparative advantage in Designs 1 and 2, relative to Hitchner modified by 112RRQRRF117 (SEQ ID NO: 7) and mutated only by L289A. There are more than 6000+Replication Competent stable wild NDV strains in the NCBI data base. These strains do not exhaust all the possible stable RC strains, but it is striking that none of these 6000+NCBI NDV strains includes the L289A mutation. The L289A mutation alters part of a conserved , structurally significant heptad L repeat sequence. Similar sequences are common to all wild fusion proteins (Dutch, R E, et al., Biosci. Rep. 20, 597 (2000)). By contrast, all the mutations used in the present synthetic Designs can be found isolated from other mutations in several of the 6000+stable wild strains in the NCBI data base. From this condition it is expected that the Replication Competency (RC) of the strains designed herein using only primers on wild strains should be maintained and even superior to that for strains that include the L289A mutation.

Sequence information for certain of the designs described above is set forth below:

For convenience an example of the already specified NDV Design sequences is:

Design 1: Hitchner F modified by 112RRQRRF117 (SEQ ID NO: 7)
and mutated by (V402A, N403D), R153L, and (Y346F, C347F, T348S).
A complete cDNA clone of the Newcastle disease virus (NDV)
strain Hitchner B1 was constructed by Nakaya T; Cros J; Park MS;
Palese P, JOURNAL OF VIROLOGY 75, 11868-11873 (2001).
(SEQ ID NO: 2)
MGSRPSTKIPAPMMLTIRVALVLSCICPANSIDGRPLAAAGIVVTGDKAVNIYTSSQTGS
IIVKLLPNLPKDKEACAKAPLDAYNRTLTTLLTPLGDSIRRIQESVTTSGGRRQRRFIGA
IIGGVALGVATAAQITAAAALIQAKQNAANILLLKESIAATNEAVHEVTDGLSQLAVAVG
KMQQFVNDQFNKTAQELDCIKIAQQVGVELNLYLTELTTVFGPQITSPALNKLTIQALYN
LAGGNMDYLLTKLGIGNNQLSSLIGSGLITGNPILYDSQTQLLGIQVTLPSVGNLNNMRA
TYLETLSVSTTRGFASALVPKVVTQVGSVIEELDTSYCIETDLDLFFSRIVTFPMSPGIY
SCLSGNTSACMYSKTEGALTTPYMTIKGSVIANCKMTTCRCADPPGIISQNYGEAVSLID
KQSCNVLSLGGITLRLSGEFDVTYQKNISIQDSQVIITGNLDISTELGNVNNSISNALNK
LEESNRKLDKVNVKLTSTSALITYIVLTIISLVFGILSLILACYLMYKQKAQQKTLLWLG
NNTLDQMRATTKM
Design 2: Hitchner F modified by 112RRQRRF117 (SEQ ID NO: 7)
and mutated by L306S and Q451L. A complete cDNA clone of the
Newcastle disease virus (NDV) strain Hitchner B1 was
constructed by Nakaya T; Cros J; Park MS; Palese P, JOURNAL
OF VIROLOGY 75, 11868-11873 (2001).
(SEQ ID NO: 3)
MGSRPSTKIPAPMMLTIRVALVLSCICPANSIDGRPLAAAGIVVTGDKAVNIYTSSQTGS
IIVKLLPNLPKDKEACAKAPLDAYNRTLTTLLTPLGDSIRRIQESVTTSGGRRQRRFIGA
IIGGVALGVATAAQITAAAALIQAKQNAANILRLKESIAATNEAVHEVTDGLSQLAVAVG
KMQQFVNDQFNKTAQELDCIKIAQQVGVELNLYLTELTTVFGPQITSPALNKLTIQALYN
LAGGNMDYLLTKLGIGNNQLSSLIGSGLITGNPILYDSQTQLLGIQVTLPSVGNLNNMRA
TYLETSSVSTTRGFASALVPKVVTQVGSVIEELDTSYCIETDLDLYCTRIVTFPMSPGIY
SCLSGNTSACMYSKTEGALTTPYMTIKGSVIANCKMTTCRCVNPPGIISQNYGEAVSLID
KQSCNVLSLGGITLRLSGEFDVTYQKNISILDSQVIITGNLDISTELGNVNNSISNALNK
LEESNRKLDKVNVKLTSTSALITYIVLTIISLVFGILSLILACYLMYKQKAQQKTLLWLG
NNTLDQMRATTKM

Virus Specification

Using the previously described Accelerated Method for Discovering and Designing Optimal Viral Proteins one can discover two groups, A and B of mutations based on hybridization with primers that greatly improve Q of Hitchner F compared to unmutated Hitchner F. Group A has seven sets can be combined in many ways, including all together, to obtain oncolytically improved viruses. The seven A sets are mutations by AI: R153L, and /or AII:(L306S, R312K) and/or AIII: (Y346F,C347F,T348S) and/or AIV: (L282I, V287I, L289S) and/orAV: (M384A) and/or AVI: (A159T, N1621) and/or AVII Y337H:. Taken altogether the improvement of smoothness predicted for mutations A(I-VI) is about 11 times that of L289A (Ebert). Group B has four sets that can be combined in many ways, including all together, to obtain oncolytically improved viruses. The four B sets are mutations by BI: (Q451L, D452N, I457V) and/or BII: (L306S, R312K) and/or BIII: (L282I, V287I, L289S) and/or BIV: Y337H. Taken altogether the improvement of smoothness predicted for mutations B(I-IV) is about 5 times that of L289A (Ebert). Mutations from groups A and B can also be combined. These primer-based mutations are also described in Tables I and II.

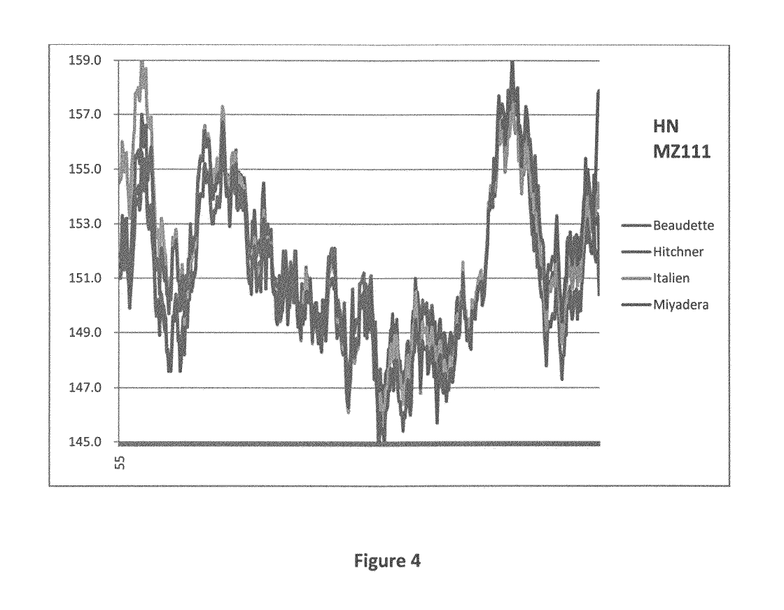

TABLE I
Smoothing Hitchner F MZ 111 with widely spaced single or small group
selected and engineered mutations, taken from the indicated Uniprot
sources. L289A reduces roughness by only 5%, whereas D1 reduces
roughness by 31% and D5 by 44%. Even the best single mutation
smoothes by only 12%, so the overall D5 smoothing by 44% occurs by
fine-tuning across the entire protein. The last line contains Y337H, a
mutation from a waterfowl strain ABU92933. It reduces roughness by
49%, about 10 times larger than L289A. Table I shows the progression
of NDV F glycoproteins from the Uniprot “Gold Star” reference
proteins to Hitchner (smallest 1/Q of these proteins) to several design
proteins shown in FIG. 4. Large improvements are obtained with only a
few RC mutations obtained by the method shown in FIG. 1, with smaller
improvements (“diminishing returns”) with additional RC mutations.

| Mutation | Source | MZ111 |
|---|---|---|
| None | Hitch | 11.28 |
| α R153L | Queen | 9.93 |
| β L306S, R312K | Miyad | 9.95 |
| γ Y346F, C347F, T348S | Queen | 10.08 |
| δ L282I, V287I, L289S | Ital | 10.38 |
| ε M384A | Ital | 10.53 |
| ζ A159T, N162I | Aus-V | 10.45 |
| D1 = α + β + γ | | 7.73 |
| D2 = D1 + δ | | 7.22 |
| D3 = D2 + ε | | 6.62 |
| D5 = D3 + ζ | | 6.36 |
| D6 = D5 + Y337H | | 5.71 |

TABLE II
Smoothing Hitch F KD 111 with widely spaced engineered small group
mutations. L289A smoothes KD111 by only 5%, whereas D4 smoothes
by 25%. The maximum reduction in roughness on the KD scale shown
here is only half that on the MZ scale shown in Table I, possibly because
of better resolution of the MZ scale.

| Mutation | Source | KD111 |
|---|---|---|
| None | Hitch | 4.92 |
| θ Q451L, D452N, I457V | Miyad | 4.03 |
| β L306S, R312K | Miyad | 4.48 |
| δ L282I, V287I, L289S | Ital | 4.55 |
| K4 = θ + β + δ | | 3.71 |
| K5 = K4 + Y337H | | 3.70 |

Using the previously described Accelerated Method for Discovering and Designing Optimal Viral Proteins one can discover eight primer-based mutations that greatly improve smoothness of the Beaudette C/45 FIN strain, which is smoother than the Hitchner/47 HN strain. The eight mutations of the Beaudette C/45 strain are: A155E, G169R, T188S, A271V, E293G, S351P, N445D, and (L32S, T39I, V41A, S43A, V45A, G49E). By choosing suitable combinations of these mutations one can optimize the enveloped fusogenic HN-F interaction. A complete cDNA clone of the Newcastle disease virus (NDV) vaccine strain Beaudette C/45 was constructed by Krishnamurthy S; Huang Z H; Samal S K, VIROLOGY 278, 168-182 (2000).

(SEQ ID NO: 4)
MDRAVSQVALENDEREAKNTWRLIFRIAILLLTVVTLATSVASLVYSMGASTPSDLVGIP
TRISRAEEKITSALGSNQDVVDRIYKQVALESPLALLNTETTIMNAITSLSYQINGAANN
SGWGAPIHDPDFIGGIGKELIVDDASDVTSFYPSEFQEHLNFIPAPTTGKGCTRIPSFDM
SATHDCYSHNVILSGCRDHSHSHQYLALGVLRTTATGRIFFSTLRSISLDDTQNRKSCSV
SATPLGCDMLCSKVTGTEEEDYNSAVPTLMVHGRLGFDGQYHEKDLDVTTLFGDWVANYP
GVGGGSFIDGRVWFPVYGGLKPNSPSDTVQEGKYVIYKRYNNTCPDEQDYQIRMAKSSYK
PGRFGGKRIQQAILSIKVSTSLGEDPVLTVPPNTVTLMGAEGRILTVGTSHFLYQRGSSY
FSPALLYPMTVSNKTATRHSPYKFNAFTRPGSPPCQASARCPNSCVTGVYTDPYPLIFYR
NHTLRGVFGTMLDSEQARLNPTSAVFDSTSRSRITRVSSSSTKAAYTTSTCFKVVKTNKT
YCLSIAEISNTLFGEFRIVPLLVEILKNDGVREARSG

The NDV attachment envelope protein FIN oncolytic art is less studied than that of the fusogenic protein F. Relative to the Hitchner/47 FIN strain, among the 13 Uniprot reference “Gold Star” NDV FIN wild sequences, the Q=S(MZ)S(KD) of the Beaudette C/45 FIN strain (P32884) is smoothest (17% smoother than Hitchner/47 HN). The Q of the present FIN a hybrid strain is 54% smoother than the Q of Hitchner/47 FIN strain. The present FIN a hybrid strain starts from the Beaudette C/45 strain (P32884). It employs the mutations A155E, S170K, [Y185D T1885], E256G, A271V, E293G, S315P, D342N, L438R, T443K, and I453P. The Q smoothness can be tuned between 17% and 54% of the Hitchner/47 FIN strain by using combinations of these mutations. These mutations separately increase smoothness as follows: A155E (4%), S170K(3%), [Y185D T1885](10%), E256G(3%), A271V(6%), E293G(7%), S315P(3%), D342N(4%), L438R(6%), T443K (6%) and I453P(7%). By choosing suitable combinations of these mutations one can optimize the enveloped fusogenic HN-F activated interaction.

One can also use the Method shown in FIG. 1 to derive the hybrid HN β strain from GenBank ABZ80390. The same primer-based hypermutations, A155E, S170K, [Y185D T1885], E256G, A271V, E293G, S315P, D342N, L438R, T443K, and I453P improve the ABZ80390 smoothness by different amounts compared to the Beaudette C/45 strain, but the final smoothness of the β hybrid strain differs from that of the a hybrid strain by only 2%. The two α, β initial strains differ by 10 site mutations, which is about 2% of 577, the number of sites. Only the Method shown in FIG. 1 can design such multiply hypermutated RC viral proteins compatible with the NCBI data base. This is why the prior state of the art stopped with the single mutation L289A not derived by primer-based mutagenesis.

TABLE III
Rank-ordered smoothed Beaudette HN MZ 111 with widely spaced single
or small group mutations, taken from the indicated Uniprot or NCBI
sources. The best single hybrid mutation α smoothes by only 10%,
so the overall D6 hybrid smoothing by 30% occurs by fine-tuning across
the entire protein.

| Mutation | Source | MZ111 |
|---|---|---|
| None | Beaudette | 6.06 |
| α Y185D, T189S | AAC55043 | 5.44 |
| β E293G | La Sota | 5.61 |
| γ N445D | Ulster | 5.64 |
| δ A271V | D26 | 5.67 |
| θ V41A, S43A, V45A, G49E | Ulster | 6.00 |
| D5 = α + β + γ + δ | | 4.35 |
| D6 = D5 + θ | | 4.23 |

TABLE IV
Rank-ordered smoothed Beaudette HN KD 111 with widely spaced single
or small group hybrid mutations, taken from the indicated Uniprot or
NCBI sources. The best single hybrid mutation θ smoothes by only
7%, so the overall D7 hybrid smoothing by 15% occurs by fine-tuning
across the entire protein.

| Mutation | Source | KD111 |
|---|---|---|
| None | Beaudette | 21.8 |
| θ V41A, S43A, V45A, G49E | Ulster | 20.3 |
| β E293G | La Sota | 20.4 |
| δ A271V | D26 | 20.9 |
| γ N445D | Ulster | 21.5 |
| α Y185D, T189S | AAC55043 | 21.6 |
| D7 All above | | 17.8 |

### References

Tisoncik Jennifer R et al., Journal of General Virology Volume: 92 Pages: 2093-2104 (2011).

Ebert, O. et al., 2010 Engineered Newcastle Disease Virus as an Improved Oncolytic Agent Against Hepatocellular Carcinoma Molecular therapy 18 275-284. Also U.S. patent application Ser. No. 12/520,571.

Collins, M. S., et al., Deduced amino acid sequences at the fusion protein cleavage site of Newcastle Disease Viruses Showing Variation in Antigenicity and Pathogenicity Archives of Virology Volume 128 363-370

1993Phillips J C 2011 arXiv:1101.2923. Protein Adaptive Plasticity and Night Vision.

Park M S; Steel J; Garcia-Sastre A; Palese P Engineered viral vaccine constructs with dual specificity: Avian influenza and Newcastle disease PNAS. (USA) 103 8203-8208 (2006)

While the invention has been described in detail and with reference to specific examples thereof, it will be apparent to one skilled in the art that various changes and modifications can be made therein without departing from the spirit and scope thereof

What is claimed is:

1. A method for inducing regression of a tumor in a subject in need thereof comprising administering to the subject a pharmaceutical composition comprising a therapeutically effective amount of an isolated, recombinant oncolytic strain of Newcastle Disease Virus (NDV) wherein at least one of the sequences encoding the F and/or HN proteins have been genetically altered to enhance in vivo stability and/or oncolytic properties when compared to sequences lacking said alterastions, said altered NDV sequences being effective to induce lysis of targeted tumor cells said virus being present in a pharmaceutically acceptable carrier.

2. The method of claim 1 comprising a nucleotide sequence selected from the group consisting of
a) F Hitchner Designs 1-6 (Table I) and/or Designs θ, β, γ, K4, K5 (Table II)
b) HN Beaudette Designs α, β, γ, δ, θ (Tables III and IV)

3. The method according to claim 2, wherein the tumor is selected from the group consisting of prostate carcinoma, colon adenocarcinoma, cervical carcinoma, endometrial carcinoma, bladder carcinoma, Wilm's tumor, fibrosarcoma, osteosarcoma, melanoma, synovial sarcoma, epidermoid carcinoma, pancreas carcinoma, endocrine system carcinoma, astrocytoma, oligodendroglioma, menigioma, neuroblastoma, glioblastoma, ependyoma, Schwannoma, neurofibrosarcoma, neuroblastoma, and medullablastoma.

4. The method according to claim 3, wherein the tumor is glioblastoma.

5. The method according to claim 1, wherein administering the pharmaceutical composition is selected from the group consisting of parenteral, oral, rectal, vaginal, topical, intranasal, inhalation, buccal, or ophthalmic administration.

6. The method according to claim 1, wherein administering the pharmaceutical composition is selected from the group consisting of intraperitoneal injection, intraarterial injection, intralesional injection into the tumor, intralesional injection adjacent to the tumor, and intraarterial infusion.

7. The method according to claim 1, wherein the therapeutically effective amount of the isolated NDV is a daily dose from about 1×10⁸ to about 5.5×10¹³ EID₅₀.

8. The method according to claim 1, wherein the therapeutically effective amount of the isolated NDV is a daily dose of about 1.1×10¹⁰ EID₅₀.

9. The method according to claim 1, wherein administering the pharmaceutical composition is determined by monitoring the level of a cancer biomarker selected from the group consisting of miR-18a for pancreatic cancer, miR-92a for colon cancer, miR-141 for prostate cancer and mir-122 and miR-192 for liver cancer.

10. The method according to claim 1, wherein the dosage cycle administration comprises administering a daily dose of the pharmaceutical composition for five successive days followed by a halt of administration.

11. The method according to claim 1, wherein the dosage cycle administration is performed at least once.

12. The method according to claim 1 wherein the dosage cycle administration is performed at least twice.

13. The method according to claim 1, further comprising administering a maintenance dose of the pharmaceutical composition at least once a week.

14. The method according to claim 12, wherein the maintenance dose is administered twice a week.

15. The method according to claim 1, wherein the subject is unresponsive to at least one anti-cancer therapy.

16. The method according to claim 1, wherein the subject is unresponsive to at least one anti-cancer therapy selected from the group consisting of tumor resection, radiotherapy and chemotherapy.

17. The method according to claim 1, wherein the therapeutically effective amount of the isolated strain of NDV is a daily dose of about 1×10⁸ to about 5.5×10¹³ EID₅₀.

18. The method according to claim 1, wherein administering the pharmaceutical composition comprises a dosage cycle administration.
